(19) United States
(12) Patent Application Publication
(10) Pub. No.: US 2005/0175579 A1
(43) Pub. Date: Aug. 11, 2005

(54) Methods and compositions for the treatment of inflammation

(76) Inventor: Michael M. Koganov, White Plains, NY (US)

Correspondence Address: TROUTMAN SANDERS LLP, BANK OF AMERICA PLAZA, SUITE 5200, 600 PEACHTREE STREET , NE, ATLANTA, GA, 30308-2216, US

(21) Appl. No.: 11/055,304

(22) Filed: Feb. 10, 2005

Related U.S. Application Data
(60) Provisional application No. 60/543,145, filed on Feb. 10, 2004.

(51) Int. Cl. A61K 38/46; A61K 31/785

(52) U.S. Cl. 424/78.27; 424/94.65

(57) ABSTRACT

The present invention comprises methods and compositions for the treatment and prevention of inflammatory conditions. The compositions comprise polymers and copolymers that are effective in modulating the activity of enzymes associated with inflammatory conditions. The methods comprise administration of effective amounts of such compositions to treat or prevent inflammatory conditions to sites of inflammation or potential inflammation.

### RELATED APPLICATIONS

The present invention claims the priority of U.S. Provisional Patent Application 60/543,145, filed Feb. 10, 2004, which is herein incorporated in its entirety.

### TECHNICAL FIELD

The present invention relates to compositions and methods for treating chronic and acute inflammatory conditions. In particular, the present invention is directed to compositions that modulate enzymes and methods of treatment using the same.

### BACKGROUND OF THE INVENTION

Chronic and acute inflammatory conditions form the basis for diseases affecting all organ systems including, but not limited to, many skin reactions, allergic reactions, asthma, lung diseases or responses, kidney diseases, acute inflammatory diseases, vascular inflammatory disease, chronic inflammation, atherosclerosis, immune related diseases, angiopathy, myocarditis, nephritis, Crohn's disease, wound healing, arthritis, type I and II diabetes and associated vascular pathologies. The incidence of these inflammatory conditions is on the rise in the population.

While inflammation in and of itself is a normal immune response, chronic inflammation leads to complications and ongoing system damage due to the interactions of cellular factors such as enzymes and cytokines. Chronic inflammation causes differing responses in different tissues, such as responses in skin leading to psoriasis or chronic dermatitis, or responses in endothelial tissue resulting in vascular complications. Coronary artery, cerebrovascular and peripheral vascular disease resulting from atherosclerotic and thromboembolic macroangiopathy are causes of mortality in chronic inflammatory diseases. The outcome of chronic inflammation can be viewed as a balance between inflammation-caused injury and repair.

In general it is believed that inflammation is a response of vascularized tissue to sublethal injury. The duration of inflammation leads to the classification as either acute or chronic. Inflammation is a homeostatic response designed to destroy or inactivate invading pathogens, remove waste and debris, and permit restoration of normal function, either through resolution or repair. Inflammatory processes appear to have shared pathways with angiogenesis and its processes in some reactions, and in others are independent of each other.

What is needed are compositions and methods that are directed to treating inflammatory conditions and that are capable of modulating cellular components triggered by inflammatory responses or components that are the triggering agent for inflammation.

### SUMMARY OF THE INVENTION

The present invention comprises compositions and methods for treating biological conditions, particularly related to inflammatory diseases, which are capable of affecting all organ systems including, but not limited to, many skin reactions, allergic reactions, asthma, lung diseases or responses, kidney diseases, acute inflammatory diseases, vascular inflammatory disease, chronic inflammation, atherosclerosis, immune related diseases, angiopathy, myocarditis, nephritis, Crohn's disease, wound healing, arthritis, type I and II diabetes and associated vascular pathologies.

In particular, the present invention comprises compositions comprising polymers capable of modulating the activity of enzymes associated with inflammation. An aspect of the compositions of the present invention comprises acrylic acid polymers or copolymers, including, but not limited to polymers and copolymers commonly known as carbomers and acrylates. Prior to the findings of the present invention, and currently, these polymers are widely used as thickeners, emulsifiers and gel-forming cosmetic formulation aid ingredients. The polymers and copolymers are thought to be inert and pose no danger of toxic effects. In the personal care items industry, acrylic acid polymers are regarded as commodity polymers used as structure-forming ingredients.

The present invention is directed to methods of affecting inflammatory responses and inflammation-related diseases and pathologies by administering the compositions of the present invention. The compositions of the present invention function to modulate the activity of enzymes involved in inflammation-related diseases and pathologies. The compositions of the present invention may modulate enzyme activity in a specific or non-specific manner. The methods comprise administration of such compositions in efficacious modes for treatment or prevention of particular inflammatory conditions.

### DESCRIPTION OF THE FIGURES

FIG. 1 is a graph showing elastase inhibitory activity of selected Acritamers®.

FIG. 2 is a graph showing elastase inhibitory activity of Acritamer® 501ER and Carbopol® ETD 2020.

FIG. 3 is a graph showing elastase inhibitory activity of Acritamer® 505E and Carbopol® 980.

FIG. 4 is a graph showing elastase inhibitory activity of Acritamer® 940 and Carbopol® 940.

FIG. 5 is a graph showing the effect of Carbopol® ETD 2020 and MDI Complex on the activity of MMP-9.

### DETAILED DESCRIPTION

The present invention is directed to compositions and methods for treatment and prevention of inflammatory conditions. The compositions of the present invention comprise polymer or copolymers that are capable of modulating the activity of enzymes involved in inflammatory conditions. The methods of the present invention comprise administering such compositions to persons or animals having an inflammatory condition in amounts effective to modulate the activity of enzymes involved in the inflammatory condition or administering the compositions in amounts effective to modulate the activity of enzymes to prevent the occurrence of an inflammatory condition. The methods and compositions of the present invention are effective in both acute and chronic inflammatory conditions.

Aspects of the compositions of the present invention comprise polymers and copolymers. An example of the polymers and copolymers of the compositions of the present invention comprise acrylic acid based polymers or copolymers (AAP). Most acrylic acid polymer products, primarily used for personal care products, are produced or distributed by several companies (Table 1).

TABLE 1
Leading Companies and AAP Products.

| Trademarks | Company Name | Headquarters |
|---|---|---|
| Carbopol ®, Pemulen ®, | Noveon, Inc. | Brecksville, OH |
| Noveon ® | | |
| Acritamer ® | RITA Corporation | Crystal Lake, IL |
| Acrisint ® | 3V-Sigma | Weehawken, NJ |
| Aqupec ® | Sumitomo Seika | Osaka, Japan |
| | Chemicals Company, Ltd. | |
| Thixol ® 100C | Coatex | Caluire, France |
| Hypan ® | Kingston Hydrogels | Dayton, NJ |
| Acrysol ® ASE-75, | Rohm & Haas Company, | Philadelphia, PA |
| Acumer ® 1510 | Inc. | |
| Sanwet ® | Hoechst Celanese Corp | Portsmouth, VA |
| Hoe S 3915 | Hoechst | Frankfurt am |
| | Aktiengesellschaft | Main, Germany |

Many different types of AAPs are produced, and all AAPs that are capable of modulating the activity of enzymes involved in inflammatory conditions and processes are contemplated by the present invention. For example, AAPs can be linear polymers of acrylic acid, or polymers cross-linked with polyalkenyl ethers or divinyl glycol or other cross-linkers. It has been reported that when these AAPs have been polymerized under the same conditions and using the same recipe as the cross-linked grades, but without the cross-linked monomer, the weight average molecular weights are in the order of about 500,000. [1] The molecular weight of cross-linked polymers is in the billions. There are two major types of cross-linked polymers:
a) homopolymers which are polymers of acrylic acid cross-linked, for example with allyl sucrose or allylpentaerythritol,
b) copolymers which are polymers of acrylic acid modified by long chain (C10-C30) alkyl acrylates, and cross-linked, for example with allylpentaerythritol. The general structures of two most frequently used acrylic homopolymers Carbopol® and copolymer Pemulen® are presented below.

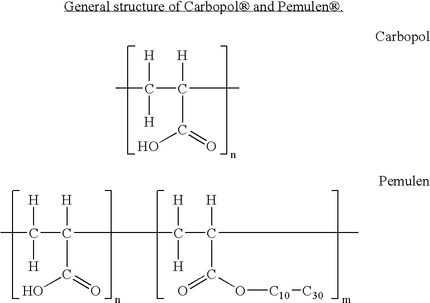

Although linear acrylic acid polymers are soluble in polar solvents, such as water, cross-linked polymers do not dissolve in water, instead they swell. When used in cosmetic formulations, a solution of cross-linked polymers with a concentration of up to 1%, no significant swelling occurs until the cross-linked polymers are partially neutralized with an appropriate base to form a salt. When this salt dissolves and ionizes, the cross-linked polymers swell into an effective thickening form [3] that are currently used as inert ingredients in many topical applications such as creams or sunscreens.

The backbone of acrylic acid homopolymers is the same and the main difference between polymers is related to cross-link density and molecular weight, rather than that type of monomer that is used as the cross-linking agent. With very minor adjustments in the cross-linker density, one can produce a large number of AAP products similar in gross molecular structure but varying in application properties, for example, viscosity. Cross-link density can be varied by minor shifts in position of the cross-linker on the acrylic backbone. Noveon's literature [2] states that “because the actual cross-linker itself has little, if any, effect on the biological properties of a particular carbopol resin, the Cosmetic, Toiletries and Fragrance Association (CTFA) has adopted a family monograph, “carbomer”, for the Carbopol® homopolymers resins”. It should be noted that, the term “biological properties” used in this publication means “biological inertness”, as prior to the present invention, it was believed that these polymers had no biological activity.

Investigations on the effect of some of the AAPs on enzyme activity have shown confusing and mixed results. Although biological inertness is claimed as one of the fundamental properties of carbomer use for personal care applications, some selected acrylic acid polymers, which are used for oral drug delivery, were shown to inactivate trypsin in vitro [4]. Lueβen et al investigated the effect of Carbopol® 934P and polycarbophil PCP Noveon® AA1 on trypsin activity and found the apparent effect the polymers had on the enzyme was due to the polymers absorbing the calcium ions and that the lack of calcium changed the secondary structure of the enzyme, thus inactivating the enzyme. This is not enzyme inhibition, but merely interference with the ability of the enzyme to bind cofactors in the environment.

Others [5] have studied nanoparticles composed of one of two polymers, polyacrylamide and poly(isobutyl cyanoacrylate) for the oral delivery of two peptides, human calcitonin (hCT) and insulin.

Bai et al [6, 7] studied the ability of Carbopols® 934P, 971P and 974P to impede the degradation action of the enzymes trypsin and chymotrypsin on human calcitonin, insulin, and insulin-like growth factor I. In vitro studies showed that the presence of the polymers caused a reduction in the pH of the incubation media to a pH below the optimum pH of the pancreatic enzymes. The enzymes will not function below the optimum pH. In vivo data provided no evidence of any effect of the tested Carbopols® on trypsin and chymotrypsin activities.

Modifications of polymers has also led to unclear results of activities. One study [8] found that both non-modified and modified acrylic acid polymers demonstrated only ion binding type of inhibition. Another study, [9], investigated the activity of modified Carbopol 974P on aminopeptidase N in vitro. Carbopol 974P was covalently linked to L-cysteine by carbodiimide linkage. Aminopeptidase N needs Zn²⁺ for activity, [10] and thus, inhibition of this enzyme could be due to the cation-binding as seen by Lueβen et al [4].

Prior to the present invention, the activities of the polymers and copolymers of the present invention with enzymes involved in inflammatory processes were not known in the public domain. In particular, the activities are not known for specific methods of treatment or prevention. For example, it is currently thought that the polymers and copolymers of the present invention are inert, and would not be beneficial for treatment or prevention of biological conditions. The acrylic acid polymers are currently believed to be only biologically neutral structural ingredients. It is believed that the stratum corneum is composed of dead and dying skin cells and that the high molecular weight acrylic acid polymers, which contain many negatively charged polar groups, are not capable of penetrating through stratum corneum to create any interactive effect. Thus the teaching prior to the present invention is that AAPs have no ability to produce any significant impact on metabolism of living skin tissue.

Recent investigations have found that there is enzymatic activity associated with skin, and is found when there has been damage, such as in an inflammatory response or condition. One enzyme that has been investigated is human leukocyte elastase (HLE).[13] HLE is a broad spectrum serine protease derived from neutrophils and macrophages and is found on the human skin surface. A large increase in HLE activity was found in the lesional skin of psoriasis (31 times), allergic contact dermatitis (55 times), and atopic dermatitis (35 times), but not in uninvolved skin of diseased patients. The presence of proteolytically active HLE in diseased epidermis suggests a pathophysiological role of this enzymatic activity in psoriasis, contact dermatitis, and atopic dermatitis. HLE has been found to induce proliferation of keratinocytes in concentrations of the enzyme that are found on the skin surface of psoriasis lesions [14]. This may indicate an explanation for the epidermal hyperproliferation observed in psoriasis.

Another skin-related enzyme, stratum corneum chymotryptic enzyme (SCCE) a serine proteinase expressed by keratinocytes in the epidermis, was characterized by Skytt et al [15]. It was suggested that the enzyme may catalyze the degradation of intercellular cohesive structures in the conified layer of the skin in the continuous shedding of cells from the skin surface. The presence of SCCE and a mature form of cathepsin D was also shown by Horikishi et al [16].

It has also been demonstrated [17] that another key cell surface enzyme, neutral endopeptidase (NEP), is involved in processes on the skin surface. This zinc-containing enzyme, which plays an active role in degradation of substance P, is produced by keratinocytes and may terminate the proinflammatory and mitogenic actions of neuropeptides on the surface of normal skin and especially wounded skin. NEP on the skin surface of diabetic wounds was described by Ludolph-Hauser et al [18].

During skin inflammation, human neutrophils release not only HLE, but additionally at least the proteinase, cathepsin G. [19] These enzymes are activated in diabetic wounds and repair of these wounds requires inhibition of both HLE and cathepsin G. The levels of matrix metalloproteinase (MMP) are elevated in chronic ulcers and these enzymes are found in cells underlying the non-healing epithelium. [20] Other enzymes have been found to be present naturally within the epidermis: cathepsins B1 and D, endoproteinase, nonspecific protease and thermolysine protease. [21-23].

The integrity of stratum corneum and other layers of skin is frequently destroyed as a result of skin inflammations, allergic reactions, wounds, ulcers and infections. This disturbance of the skin layers can cause redistribution of endogenous proteinases between epidermis and skin surface. The extent of destruction of the layered structure of skin may be due to introduction of these enzymes to layers where they are not usually found and the resultant activity of these enzymes, possibly triggered by factors released due to the inflammation and initial change in structure, such as a wound. There may also be resident enzymes in the layers of skin and the numbers of them are increased, and/or the activity levels are increased in response to the injury to the site or presence of inflammatory factors. It is generally agreed that elevated levels of proteolytic enzymatic activities is an indication of inflammation injury and its inhibition initiates an anti-inflammatory response. For example, at inflammatory sites in the skin, neutrophil elastase is generally present at the highest concentration and is the most active proteinase against the widest variety of connective tissue components, including elastin

Microorganisms present on the skin surface have their own enzymes and the complete picture of all the possible factors and cellular participants may be quite complex. Average counts of bacteria per cm² of skin, depending of the part of the body, including forehead and nose, range from 710 to 3,900,000. Others have found the average count on forearms of 14,000 to 87,000 bacteria per cm² depending on the type of skin. [25] This enzymatically rich bacterial flora produces proteinases and phospholipases which can contribute to the activities on the stratum corneum surface.

TABLE 2
Localization of Enzymatic Activities.

| | Localization of | |
|---|---|---|
| Enzyme | Enzyme | References |
| Cathepsin B | Skin Surface | [15] |
| Cathepsin D | Skin Surface | [21] |
| Cathepsin G | Skin Surface | [21] |
| Endoproteinase | Skin Surface | [23] |
| HLE | Skin Surface | [13, 14, 19] |
| MMP | Skin Surface | [20] |
| NEP | Skin Surface | [17, 18] |
| Nonspecific protease | Skin Surface | [22] |
| SCCE | Skin Surface | [15, 16] |
| Thermolysine | Skin Surface | [23] |
| protease | | |

The present invention comprises compositions of linear polymers or copolymers that affect or modulate the activity of enzymes. The terms polymers and copolymers are used interchangeably herein, and polymer includes copolymer. An embodiment of the present invention comprises compositions that modulate the enzyme activities associated with inflammatory conditions. An aspect of the present invention comprises compositions that are effective in modulating the activity of enzymes associated with inflammatory conditions or reactions of the skin and integumentary system of humans and animals. Enzymes that are affected by the compositions and methods of the present invention include those involved in inflammatory conditions including, but not limited to, many skin reactions, allergic reactions, asthma, lung diseases or responses, kidney diseases, acute inflammatory diseases, vascular inflammatory disease, chronic inflammation, atherosclerosis, immune related diseases, angiopathy, myocarditis, nephritis, Crohn's disease, wound healing, arthritis, type I and II diabetes and associated vascular pathologies.

The compositions of the present invention comprise acrylic acid polymers and copolymers. A composition comprises an effective amount of an acrylic acid polymer or copolymer (referred to herein as AAP) in a pharmaceutically acceptable carrier or excipient composition. For example, a composition comprises an AAP in range of about 1 microgram to 5 g per dose or application, or a composition may comprise from about 0.001% wt to about 99% wt of one or more AAPs. Ranges of AAPs in compositions include amounts effective for treatment and prevention of inflammatory conditions, and include from about less than 0.05%, from about 0.001% wt. to less than about 0.05% wt, from about less than 0.1% wt, from about 0.001% wt to about 25% wt, from about 0.001% wt to about 15% wt, from about 0.001% wt to about 50% wt, from about 0.001% wt to about 55% wt, from about 0.001% wt to about 75% wt, from about 0.001% wt to about 85% wt, from about 0.001% wt to about 90% wt, from about 0.001% wt to about 95% wt, or about less than 0.05% wt, about less than 0.10% wt, about less than 0.5% wt, about less than 1.0% wt, about less than 5.0% wt, about less than 10.0% wt, about less than 25.0% wt, about less than 50% wt, about less than 65% wt, about less than 75% wt, about less than 80% wt, about less than 90% wt, or about less than 95% wt.

For example, for an emulsion formulation, a composition comprises 0.01% wt. of acrylates/C10-30 alkyl acrylate crosspolymer. Compositions may comprise one or more different AAPs, or mixtures of AAPs. The present invention comprises AAP such as, but not limited to, the polymers shown below.

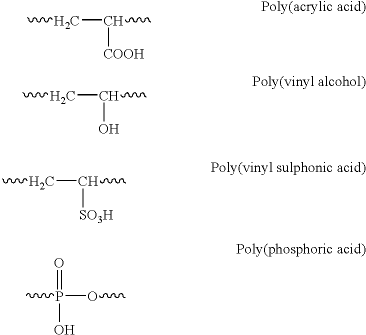

The compositions of the present invention comprise AAP polymers that can either dissolve or swell in water and form either a solution or a hydrogel. They have estimated world market around US$6 billion per year. They appear in a great variety of products and find applications in many fields including: water treatment, cosmetics, personal care products, pharmaceuticals, oil recovery, pulp and paper production, mineral processing, and agriculture, etc. The manufacture of these polymers is generally commercially implemented by various processes including aqueous solution polymerization, inverse suspension (W/O) polymerization, and inverse emulsion (W/O) polymerization, which are initiated by either thermal initiators or redox couple initiators. Among all of these polymers, poly(acrylic acid) and polyacrylamide based polymers are used in a wide range of products because they are regarded as inert.

The key to water solubility and swelling lie in positioning sufficient numbers of hydrophilic functional groups along the backbone or side chains of polymers. Some of the major functional groups that possess sufficient polarity, charge, or hydrogen bonding capability for hydration include, but are not limited to: 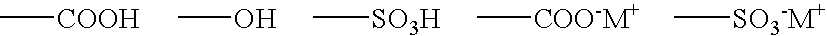
The above functional groups not only impart solubility, but also bring many useful properties like chelating, dispersing, absorption, flocculation, thickening, drag reduction and etc. to the polymers. Moreover, some of these groups can further react to form other kinds of functional groups, so the water-soluble and water-swelling polymers find extensive applications in areas including water treatment, cosmetics, personal care product, pharmaceutical, oil recovery, pulp and paper production, mineral processing, and agriculture.

The present invention comprises synthetic water soluble and water-swelling polymers. These polymers are commonly synthesized from water-soluble monomers, like: acrylic acid (AA) and its sodium salt, acrylamide (AM), hydroxyethyl methacrylate (HEMA), hydroxyethyl acrylate (HEA), vinylyyrolidone (VP), quaternary ammonium salt, like dimethyldiallyl ammonium chloride (DMDAAC) and etc. They generally follow the free radical polymerization mechanism. The synthesis is commercially implemented by various processes including aqueous solution polymerization, inverse suspension polymerization and inverse emulsion polymerization.

Solution polymerization is commonly used in the synthesis of linear, low molecular weight water-soluble polymers. Poly(acrylic acid) and its copolymers, and polyacrylamide and its copolymer with DMDAAC are polymerized in solution. In order to synthesize the high molecular weight poly(acrylic acid), polyacrylamide and their copolymers, inverse suspension/emulsion processes are used. In the solution process, the water-soluble monomers are polymerized in a homogenous aqueous solution in the presence of free-radical initiators, mostly redox couples. The solution process requires low operating costs, principally in the avoidance of materials such as organic phases and emulsifiers. Linear, high molecule weight, polyacrylamide-based polymers are commercially synthesized through inverse emulsion (W/O, 0.05-1 μm) polymerization, while the production of lightly crosslinked, poly(acrylic acid)-based polymers is generally manufactured by inverse suspension (W/O, 0.05-2 mm) polymerization. In both cases, the aqueous monomer mixture (i.e. water phase) is emulsified/suspended in an aliphatic or aromatic hydrocarbon phase (i.e. oil phase), and the size of particles strongly depends on the chemical and physical properties of the emulsifiers or dispersing agents used.

Nonlimiting examples of enzymes that are affected by the compositions of the present invention include peptide hydrolases, serine proteases, matrix metalloproteinases, collagenases, kinases, elastases and peroxydases.

Methods of the present invention comprise administration of compositions comprising polymers or copolymers that are capable of modulating the activity of enzymes involved in inflammatory conditions. Nonlimiting examples of such polymers or copolymers are included in the Examples and charts herein. Compositions of the present invention comprise polymers and copolymers including, but not limited to, linear acrylic acid-based polymers, cross-linked acrylic acid-based polymers, high molecular weight cross-linked acrylic acid-based polymers, polymers of acrylic acid cross-linked with allyl sucrose, polymers of acrylic acid cross-linked with allylpentaerythritol, polymers of acrylic acid, modified by long chain (C10-C30) acrylates, polymers of acrylic acid, modified by long chain (C10-C30) acrylates that are cross-linked with allylpentaerythritol, copolymers of acrylic acid, modified by long chain (C10-C30) alkyl acrylates, and copolymers of acrylic acid, modified by long chain (C10-C30) alkyl acrylates cross-linked with allylpentaerythritol, polymers of acrylic acid cross-linked with divinyl glycol, homopolymers of acrylic acid cross-linked with an allyl ether of penaethritol, an allyl ether of sucrose or an allyl ether of propylene, polyvinyl carboxy polymers, carbomers, copolymers of C- to C-30 alkyl acrylates and one or more monomers of acrylic acid, methacrylic acid or one of their simple esters cross-linked with an allyl ether of sucrose or an allyl ether of pentaerythritol, graft copolymers with acrylic polymer backbone and dimethylpolysiloxane side chains, hydrophilic/hydrophobic block copolymers such as ammonium acylates and acrylonitrogen copolymers, acrylic and acrylonitrogen copolymers, acrylic acid polyquaternium copolymers, polyglycols, hydrophobically modified ethylene oxide urethanes, polymers and copolymers marketed under the tradename Acusol by Rohm and Haas, and other polymers and copolymers that are capable of modulating the activity of enzymes associated with inflammatory conditions.

Other peptide hydrolases, such as gelatinase B or matrix metalloproteinase (MMP-9) acts synergistically with elastase and plays an important role in skin inflammation. It should be noted, that both MMP-9 and elastase are secreted by white blood cells (neutrophils) and these enzymes are enzymes leading to inflammation.

A composition that can inhibit both enzymes, elastase and MMP-9, would be very effective to treat or prevent inflammatory processes. Aging processes, sunburns, formation of wounds and scars have the same inflammation mechanism, which involves both MMP-9 and elastase. Thus, compositions capable of inhibiting both MMP-9 and elastase have a very wide spectrum of applications. These two enzymes work together to degrade all the components of extracellular matrix of human tissue. Elastase can inactivate the body's own inhibitory defense against MMP-9 and MMP-9 can inactivate the body's own inhibitory defense against elastase.

As used herein, modulating the activity of enzymes includes inhibition of activity and stimulation of activity, depending on the measured change. The activity change can be a change in the activity of one or more enzymes, such as an increase in turn-over of substrate; or a change in the activity of one or more enzymes that were quiescent or active prior to administration of the compositions of the present invention, such as inhibition of active enzymes which lessens the tissue destruction. A change in enzyme activity can be determined by measuring the enzyme activity or by a measurable change in the inflammatory condition. Treatment of inflammatory conditions using the compositions taught herein comprises administering the compositions in an amount effective to modulate the activity of enzymes and may comprise measurable changes in the patient, human or animal, with the inflammatory condition. For example, if the skin of a patient is undergoing an inflammatory response, treatment comprises applying a composition of the present invention to that skin, until there is a change in the appearance or function of that skin so that a skilled practitioner would no longer diagnose the skin as having an inflammatory condition, such as in the inflammatory response ceases or subsides.

Prevention of inflammatory conditions using the compositions taught herein comprises administering the compositions in an amount effective to modulate the activity of enzymes and may comprise preventing measurable changes in the patient, human or animal, with the inflammatory condition. For example, if the skin of a patient has undergone an inflammatory response previously, but is not currently undergoing such an inflammatory response, or if the patient has never undergone an inflammatory response, prevention comprises applying a composition of the present invention to that skin, prophylactically to prevent the occurrence of an inflammatory response.

Compositions of the present invention may be administered by a route which includes, but is not limited to, oral, parenteral, epidermis, surface, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal.

Methods of the present invention comprise administering an effective amount of a composition taught herein for the treatment and/or prevention of inflammatory conditions. An aspect of the invention comprises administering a composition comprising an effective amount of an AAP for treatment of inflammation of the skin.

A cosmetic or pharmaceutical composition containing effective amounts of AAPs can be effectively applied as an emulsion (lotion, cream and spray), gel or solution. Emulsions, preferably oil-in-water type emulsions, but not limited to water-in oil, water-in-silicone, triple emulsions, W/O/W or O/W/O, and microemulsions, can be utilized. Examples include AAPs that are incorporated in compositions at concentration amounts that are effective for treatment of inflammation (for example, below 0.05% wt.), but may not affect the rheological properties of composition.

Emulsions or gels may include at least one of the following additional components: emulsifier, emollient, rheology modifying agent, skin-feel additive, moisturizing agent, humectant, film former, pH adjuster/chelating agent, preservative, fragrance, effect pigment, color additive, water or any combinations thereof. Pharmaceutical excipients are known to those skilled in the art, and pharmaceutical composition components are known for compositions for use in the routes of administration taught herein.

Suitable emulsifier types include esters of glycerin, esters of propylene glycol, fatty acid esters of polyethylene glycol, fatty acid esters of polypropylene glycol, esters of sorbitol, esters of sorbitan anhydrides, esters and ethers of glucose, ethoxylated ethers, ethoxylated alcohols, alkyl phosphates, polyoxyethylene fatty ether phosphates, fatty acid amides, acyl lactylates, soaps and mixtures thereof. Emulsifiers that may be used in the compositions of the present invention include, but are not limited to sorbitan oleate, sorbitan sesquioleate, PEG-100 stearate, sorbitan isostearate, sorbitan trioleate, polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, polyglyceryl-3-diisostearate, polyglycerol esters of oleic/isostearic acid, polyglyceryl-4-oleate, polyglyceryl-4 oleate/PEG-8 propylene glycol cocoate, sodium glyceryl oleate phosphate, hydrogenated vegetable glycerides phosphate, cetearyl glucoside, cocoyl glucoside, disodium coco-glucoside citrate, disodium coco-glucoside sulfosuccinate, oleoyl ethyl glucoside, sodium coco-glucoside tartrate, or any combinations thereof. The compositions according to the present invention can also comprise lipophilic emulsifiers as skin care actives. Suitable lipohilic skin care actives include anionic food grade emulsifiers which comprise a di-acid mixed with a monoglyceride such as succinylated monoglycerides, monostearyl citrate, glyceryl monostearate diacetyl tartrate and mixtures thereof. The amount of emulsifier present in the emulsion of the present invention is preferably between 0.1 wt. % to about 20 wt. %, but most preferably between 1 wt. % to about 12 wt. % of the total weight of the composition.

The compositions of the present invention also include water or other solvents, which combined with water. Water is present in an amount preferably between 5 wt. % to about 95 wt. %, but preferably between 45 wt. % to about 90 wt. %, of the total weight of the emulsion.

The present composition may include one or more emollients. An emollient provides a softening or soothing effect on the skin surface. Suitable emollients include, but are not limited to cyclomethicone, isopropyl myristate, dimethicone, dicapryl maleate, caprylic/capric triglyceride, mineral oil, lanolin oil, coconut oil, cocoa butter, shea butter, olive oil, castor oil, fatty acid such as oleic and stearic, fatty alcohol such as cetyl and diisopropyl adipate, hydroxybenzoate esters, benzoic acid esters of C₉-C₁₅ alcohols, alkanes such as mineral oil, silicone such as dimethyl polysiloxane, ether such as polyoxypropylene butyl ether and polyoxypropylene cetyl ether, C₁₂-C₁₅ alkyl benzoate, or any combinations thereof. The total amount of emollient present in the emulsion is preferably between 0.1 wt. % to 70 wt. %, but most preferably between 0.1 wt. % to about 30 wt. %, based on the total weight of the composition.

The present composition may include one or more rheology modifying agents. Suitable rheology modifying agents for use in the compositions of the present invention include, but are not limited to, thickening agents, synthetic and natural gum or polymer products, polysaccharide thickening agents, associative thickeners, modified starch or any combinations thereof. Suitable rheological additives and stabilizers that may be used in the compositions of the present invention include synthetic and natural gum or polymer products, polysaccharide thickening agents, associative thickeners, anionic associative rheology modifiers, nonionic associative rheology modifiers, polysaccharides, polyether-1, sodium magnesium silicate, carragenan, sodium carboxymethyl dextran, hydroxyethylcellulose, hydroxypropyl cyclodextran, bentonites, trihydroxystearin, aluminum-magnesium hydroxide stearate, xantan gum, or any combinations thereof. The total amount of rheology modifying agent present in the emulsion is preferably between 0.1 wt % to 5 wt %, most preferably between 0.1 wt. % to about 2 wt. %, based on the total weight of the composition

A skin-feel additive may be also included. Skin-feel additives include, but are not limited to polymers, silicones, esters, particulates, or any combinations thereof. Preferably, the skin-feel additive is present in the emulsion in an amount about 1 wt. % to about 5 wt. %, based on the total weight of the composition.

The pH of the compositions of the present invention may be adjusted by one or more known pH adjusters and/or chelating agents. For example, sodium hydroxide, citric acid, triethanolamine, disodium ethylenediaminetetraacetic acid, or any combinations thereof are suitable pH adjusters/chelating agents that may be included in the emulsion of the present invention. An effective amount of a pH adjuster and/or chelating agent that may be included to adjust the pH of the final composition to about 3 to about 8.

A moisturizing agent, such as a humectant, may be used in the compositions of the present invention. Humectants include, but are not limited to glycerin, polyethylene glycol, polypropylene glycol, penthylene glycol, sorbitol, or any combinations thereof.

One or more moisturizing agents are optionally included in the compositions of the present invention in an amount about 1 wt. % to about 20 wt. % of the total weight of the composition.

Another component that may be used in an emulsion of the present invention is a film former agent. The film former agent is a hydrophobic material that imparts film forming and sustained release characteristics to the emulsion. One or more film formers may be present in a composition of the present invention in an amount about 1 wt. % to about 5 wt. %, based on the total weight of the composition.

Optionally, one or more preservatives and antioxidants may be included in a composition of the present invention. Examples include diazolidinyl urea, iodopropynyl butylcarbamate, chloromethylisotiazolinone, methylisothiazolinone, vitamin E and its derivatives including vitamin E acetate, vitamin C, butylated hydroxytoluene, methylparaben, propyl paraben, sodium benzoate, potassium sorbate, phenoxyethanol or any combinations thereof.

About 0.01 wt. % to about 1 wt. % of preservative and antioxidant may be included in a composition of the present invention.

The emulsion may also have other optional additives. For instance, one or more sunscreen active ingredients, fragrances, colorants, plant extract, absorbents, thickeners, salicylic acid, alpha and beta hydroxy acids, vitamins including vitamins A, C, and E, retinol, retinol palmitate, tocopherol, or any mixtures thereof, may be included in the emulsions.

Suitable for use herein are ingredients which comprise any compound, composition or mixture thereof having antiperspirant activity that may have inflammatory potential. Astringent metallic salts are preferred antiperspirant materials for use herein, particularly the inorganic and organic salts of aluminum, zirconium and zinc, as well as mixtures thereof. Particularly preferred are the aluminum and zirconium salts, such as aluminum halides, aluminum hydroxy halides, zirconyl oxide halides, zirconyl hydroxy halides, and mixtures thereof.

Also useful herein are sunscreening agents that may have inflammatory potential, like 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyidibenzoylmethane, 4-isopropyl dibenzoylmethane, 3-benzylidene camphor, 3-(4-methylbenzylidene)camphor, titanium dioxide, zinc oxide, silica, iron oxide, and mixtures thereof.

Useful pharmaceutical actives in the compositions of the present invention include inflammatory potential activators such as anti-acne keratolytics agents, such as salicylic acid, sulfur, lactic acid, glycolic, pyruvic acid, urea, resorcinol, and N-acetylcysteine; retinoids such as retinoic acid and its derivatives (e.g., cis and trans); antibiotics and antimicrobials such as benzoyl peroxide, octopirox, erythromycin, zinc, tetracyclin, triclosan, azelaic acid and its derivatives, phenoxy ethanol and phenoxy proponol, ethylacetate, clindamycin and meclocycline; sebostats such as flavinoids; alpha and beta hydroxy acids; and bile salts such as scymnol sulfate and its derivatives, deoxycholate, and cholate. Useful pharmaceutical actives in the compositions of the present invention include analgesic actives.

Analgesic actives suitable for use in the present compositions that could be benefit from the carrier compositions that include the embodiment of the invention include salicylic acid derivatives such as methyl salicylate, species and derivatives of the genus capsicum such as capsaicin and non-steroidal anti-inflammatory drugs (NSAIDS). The NSAIDS can be selected from the following categories: propionic acid derivatives; acetic acid derivatives; fenamic acid derivatives; biphenylcarboxylic acid derivatives; and oxicams. Most preferred are the propionic NSAIDS including but not limited to aspirin, acetaminophen, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen and bucloxic acid. Also useful are the steroidal anti-inflammatory drugs including hydrocortisone and the like.

Useful pharmaceutical actives in the compositions of the present invention include antipruritic drugs. Antipruritic actives preferred for inclusion in compositions of the present invention include pharmaceutically-acceptable salts of methdilizine and trimeprazine. Useful pharmaceutical actives in the compositions of the present invention include anesthetic actives. Anesthetic actives preferred for inclusion in compositions of the present invention include pharmaceutically acceptable salts of lidocaine, bupivacaine, chlorprocaine, dibucaine, etidocaine, mepivacaine, tetracaine, dyclonine, hexylcaine, procaine, cocaine, ketamine and pramoxine.

Useful pharmaceutical actives in the compositions of the present invention include antimicrobial actives (antibacterial, antifungal, antiprotozoal and antiviral drugs). Antimicrobial actives preferred for inclusion in compositions of the present invention include pharmaceutically-acceptable salts of b-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, metronidazole, pentamidine, gentamicin, kanamycin, lineomycin, methacycline, methenamine, minocycline, neomycin, netilmicin, paromomycin, streptomycin, tobramycin, miconazole and amanfadine. Antimicrobial drugs preferred for inclusion in compositions of the present invention include tetracycline hydrochloride, erythromycin estolate, erythromycin stearate (salt), amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, parachlorometa xylenol, nystatin, tolnaftate and clotrimazole.

The components of the present invention may be combined to form a stable emulsions, gel or solution. The AAP is incorporated into the water phase and later can be combined with other ingredients.

The composition is applied at least once a day to the affected area of the skin for at least one day. An example of treatment of burns and the resulting inflammation of the skin comprises applying a cream formulation composition comprising 0.01% of acrylates/C10-30 alkyl acrylate crosspolymer (see Example 4), until the skin is no longer inflamed.

It must be noted that, as used in this specification and the appended claims, the singular forms “a,” “an,” and “the” include plural referents unless the context clearly dictates otherwise.

All patents, patent applications and references included herein are specifically incorporated by reference in their entireties.

It should be understood, of course, that the foregoing relates only to preferred embodiments of the present invention and that numerous modifications or alterations may be made therein without departing from the spirit and the scope of the invention as set forth in this disclosure.

The present invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1

The AAPs used for evaluation of their effect on elastase activity, were selected from carbomers, for example polymers distributed by RITA Corporation (Acritamer®) and manufactured by Noveon, Inc. (Carbopol®). The properties and brief descriptions of selected Acrotamers® are presented in Table 3.

TABLE 3
Properties of Selected AAPs.

| | Product Definition and | | | |
|---|---|---|---|---|
| RITA Product | Description | pH* | Viscosity** | Clarity** |
| Acritamer ® 501ER | Copolymer of C-10-30 | No Data | 1.0% | No Data |
| CAS: 3906-90-50 | alkyl acrylate and one or | | 25,000-45,000 | |
| INCI: Acrylates/ | more monomers of acrylic | | 1.0% + 1.0% | |
| C-10-C30 Alkyl | acid, methacrylic acid or | | NaCl | |
| Acrylate | one of their simple esters | | 7,000-14,000 | |
| Crosspolymer | cross-linked with an allyl | | | |
| | ether of sucrose or an allyl | | | |
| | ether of pentaerythritol | | | |
| Acritamer ® 505E | Polyvinyl carboxy | 2.7 to 3.3 | 0.2% | >82% |
| CAS: 9003-01-4 | polymer. Homopolymer of | | 15,000-30,000 | |
| INCI: Carbomer | acrylic acid cross linked | | 0.5% | |
| | with ethers of | | 40,000-70,000 | |
| | pentaerythritol, an allyl | | | |
| | ether of sucrose or an allyl | | | |
| | ether of propylene | | | |
| Acritamer ® 940 | Homopolymer of acrylic | 2.7 to 3.3 | 0.2% | >80% |
| CAS: 9003-01-4 | acid cross linked with an | | 15,000-30,000 | |
| INCI: Carbomer | allyl ether of | | 0.5% | |
| | pentaerythritol, an allyl | | 40,000-70,000 | |
| | ether of sucrose or an allyl | | | |
| | ether of propylene | | | |
| Acritamer ® PNC- | Acrylic based polymer | 6.0 to 7.0 | 1.0% | No Data |
| EG*** | | | 25,000-35,000 | |
| CAS: 9003-01-4, | | | | |
| 255949-84-2 | | | | |
| INCI: Sodium | | | | |
| Polyacrylate | | | | |

*0.5% Solution
**Neutralized solution
***Active content 85-100%

Based on certain similarities between RITA's and Noveon's acrylic polymers, the following AAPs products were used for evaluation of their enzyme inhibition activity (Table 4).

TABLE 4
AAPs Products Selected for Evaluation of Inhibitory Activity.

| | | | Similar |
|---|---|---|---|
| RITA Product | Product Definition and | | Noveon |
| Information | Description | Selection | Product |
| Acritamer ® 501ER | Copolymer of C-10-30 alkyl | Highest | Carbopol ® |
| CAS: 3906-90-50 | acrylate and one of more | compatibility with | ETD 2020 |
| INCI: Acrylates/ | monomers of acrylic acid, | electrolyte | |
| C-10-C30 Alkyl | methacrylic acid or one of their | solutions | |
| Acrylate | simple esters cross-linked with | | |
| Crosspolymer | an allyl ether of sucrose or an | | |
| | allyl ether of pentaerythritol | | |
| Acritamer ® 505E | Polyvinyl carboxy polymer. | Has | Carbopol ® |
| CAS: 9003-01-4 | Homopolymer of acrylic acid | highest | 980 |
| INCI: Carbomer | cross linked with ethers of | clarity | |
| | pentaerythritol, an allyl ether of | of | |
| | sucrose or an allyl ether of | neutralized | |
| | propylene | solution | |
| Acritamer ® 940 | Homopolymer of acrylic acid | Efficient | Carbopol ® |
| CAS: 9003-01-4 | cross linked with an allyl ether | thickener | 940 |
| INCI: Carbomer | of pentaerythritol, an allyl ether | at | |
| | of sucrose or an allyl ether of | high | |
| | propylene | viscosity | |
| Acritamer ® PNC- | Acrylic based polymer | Neutralized | None |
| EG* | | form of | identified |
| CAS: 9003-01-4, | | polymer | |
| 255949-84-2 | | | |
| INCI: Sodium | | | |
| Polyacrylate | | | |

Because selected AAPs have limited and quite different swelling capabilities the following procedure was developed to equalize the conditions of samples preparation. The Acritamers® and Carbopols® polymers were all suspended in 50 mM Tris-HCl buffer, pH 7.3 by adding 6 mg of dry material slowly to 12 mL buffer while vortexing slowly. The suspensions were placed on an end-over-end rocker for 1 hour to ensure even dispersion and then placed in a 37° C. incubator for 48 hours to achieve complete dissolution. At the end of this time, there was no visible evidence of aggregates or insoluble residue in any of the preparations. These stock solutions each have an acrylic acid polymer concentration of 500 μg/mL.

### Example 2

Elastase inhibition was determined using synthetic soluble peptide substrate which is specific for human neutrophil elastase (HNE) along with a source of the enzyme activity which is derived from human inflammatory fluids. The substrate (methoxysuccinyl-Ala-Ala-Pro-Val-p-nitroanilide) was employed for these assays, and the source of HNE was a purified enzyme preparation derived from the airway secretions of patients with cystic fibrosis. Enzymatic cleavage of the substrate results in generation of increasing yellow color over time; the rate of color generation is diminished by increasing concentrations of tested samples containing inhibitory activity. Analysis of the concentration dependence of inhibition permits quantification of the potency of the inhibitory activity, expressed as that concentration of dry matter within each fraction required to achieve 50% inhibition (IC₅₀), but also provides information relating to the mode of inhibition. When the value of the inhibition constant, Kᵢ, is significantly lower than the value of IC₅₀, at least part of the mechanism of inhibition involves blocking the active site of the enzyme, i.e. “competitive” inhibition. Graphical analysis of the inhibition data also provides clues to whether the mode of inhibition is reversible or irreversible. Since neutrophil elastase has some positive physiological roles when present at controlled levels, indiscriminate use of irreversible inhibitors may compromise these normal functions of the enzyme.

The polymer stock solutions (acrylic acid polymer concentration of 500 μg/mL) were diluted into the same Tris-HCl buffer and 50 μL aliquots of the series of dilutions were added to 50 μL aliquots of a 4.5 μg/mL solution of human neutrophil elastase (HNE) in the same buffer in 96 well microplates. After mixing to ensure uniformity of distribution of polymer, elastase activity in the wells was assayed by recording the increase in optical density at 405 nm for a period of 10 minutes after addition of 50 μL aliquots of a 450 μM solution of the chromogenic substrate methoxysuccinyl-Ala-Ala-Pro-Val-p-nitroanilide in Tris buffer containing 10% DMSO (final substrate concentration=150 μM). All measurements were made using multiwell microplate reader. The observed amidolytic rates were all compared to those of control wells containing enzyme, buffer, and substrate but no polymers.

Results in the figures are expressed as percentages of the amidolytic rates of the control wells for each individual experiment. In all cases, the final concentrations of polymers indicated are in units of μg/mL.

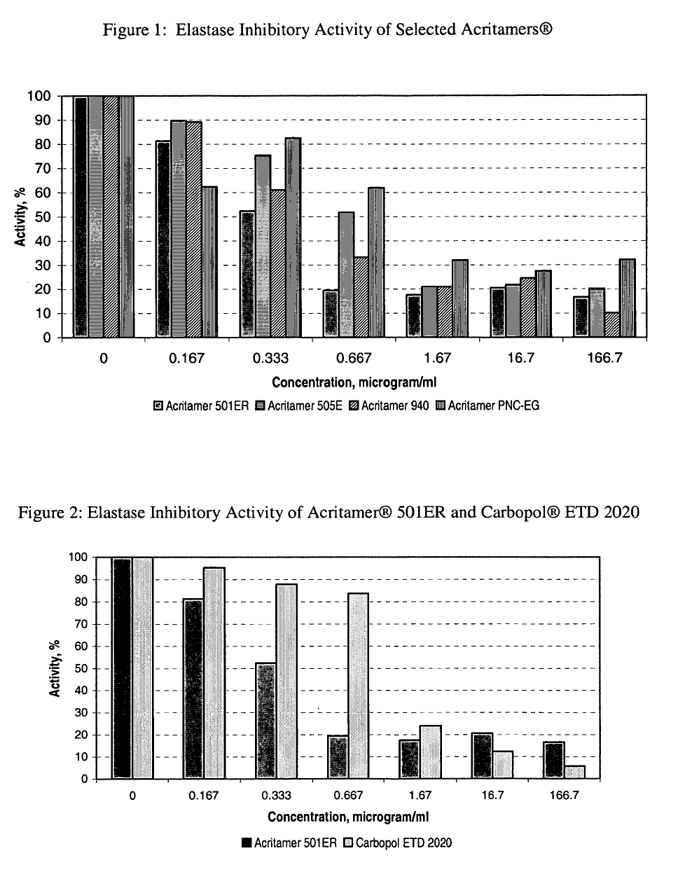

As a result of the acrylic acid polymer in-vitro evaluation, it was found that all four selected AAP products of RITA Corporation (Acritamers®) were able to demonstrate impressive elastase inhibitory activity as shown in FIG. 1.

The anti-elastase activity is decreasing in the following sequence: Acritamer® 501ER > Acritamer® 940 > Acritamer® 980 > Acritamer® PNC-EG. The differences between IC₅₀ values are quite significant. Thus the most potent inhibitory activity is associated with Acritamer® 501ER having IC₅₀=0.3 μg/ml and in three times less potent elastase inhibitory activity is associated with Acritamer® PNC-EG (IC₅₀=0.9 μg/ml). The IC₅₀ of Acritamers® 505E and 940 are in the range of 0.5-0.6 μg/ml.

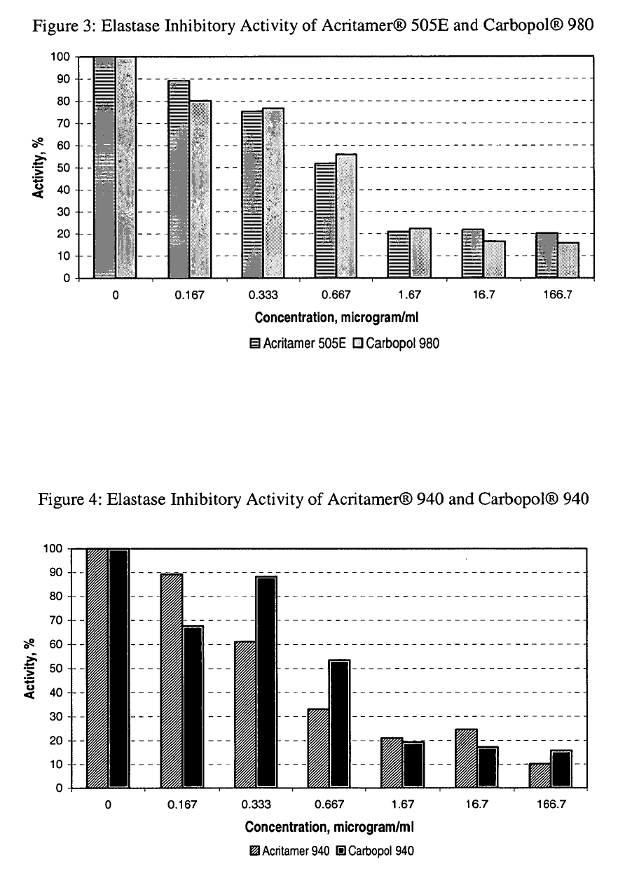

It should be noted, that AAPs manufactured by Noveon-Carbopols® also demonstrated marked enzyme inhibitory activity, although Acritamers® are somewhat more potent elastase inhibitors than the Carbopols®. The comparative results related to particular Acritamer® products with similar Carbopol® products are presented on FIGS. 2-4.

The comparison of IC₅₀ values related to all selected AAPs products provides evidence that Acritamers® are more potent elastase inhibitors than the Carbopols® (Table 5).

TABLE 5
IC₅₀ Values of Selected AAPs Products.

| RITA | IC₅₀ | Similar Noveon's | IC₅₀ |
|---|---|---|---|
| Product | μg/ml | Product | μg/ml |
| Acritamer ® 501ER | 0.3 | Carbopol ® ETD 2020 | 1.0 |
| CAS: 3906-90-50 | | | |
| Acritamer ® 505E | 0.6 | Carbopol ® 980 | 0.7 |
| CAS: 9003-01-4 | | | |
| Acritamer ® 940 | 0.5 | Carbopol ® 940 | 0.8 |
| CAS: 9003-01-4 | | | |
| Acritamer ® PNC- | 0.9 | No identified | Not applicable |
| EG* | | | |
| CAS: 9003-01-4, | | | |
| 255949-84-2 | | | |

None of the Acritamers® or Carbopols® could achieve complete inhibition of elastase activity: approximately 5-20% residual activity could still be detected at AAPs concentrations of two orders of magnitude higher than the IC₅₀ values. At high concentrations of the Carbopol® ETD 2020 approximately 95% inhibition could be achieved. The Acritamer® 940 at highest concentration could inhibit approximately 90% of enzymatic activity. The effect has been seen with another polyanionic polymer.

It was found that enzyme inhibition properties of acrylic acid polymers may depend on concentration of electrolyte. Thus at high concentration (1.0 M NaCl) inhibitory effect of AAPs is completely eliminated. Though not wishing to be bound by any particular theory, it is thought that electrostatic interaction between enzyme and polar groups of AAPs may be responsible for the inhibition of tested polymers. It should be noted, that effects of 1.0 M concentration of electrolyte is significant only for demonstrating the nature of inhibitory mechanism, since they involve the usage of nonphysiological conditions. The physiological concentration is 0.15 M, which is much lower than 1.0 M concentration of electrolyte required to eliminate the inhibitory effect of AAPs. Thus, at physiological conditions acrylic acid polymers can effectively inhibit elastase.

The elastase inhibition activity of AAPs could be compared with specific activity of acrylic acid polymer-free elastase inhibitors such as Elhibin® (Pentapharm, Switzerland). Control experiments showed that the Elhibin® (preparation containing approximately 2.5% (w/v) of active soya peptides) has IC₅₀=3.5 μg dry matter/ml. This special cosmetic ingredient is at least a 10 times less potent elastase inhibitor than Acritamer® 501ER. It is thought that Elhibin® has a predominantly non-electrostatic interaction with proteases and thus is an irreversible inhibitor of enzymes, which could create regulatory problems. It appeared that for Acritamers®, that the inhibitory effect is reversible.

### Example 3

MMP-9 was selected for next step evaluation of AAPs enzyme inhibition properties.

Interestingly, MMP-9 and Elastase have very different physico-chemical and biochemical properties. For example, MMP-9 is a complex enzyme containing 14 ions (10 Cu⁺ & 4 Zn²⁺) in the active center of the enzyme. MMP-9 consists of two peptide chains and has a molecular weight>90,000 Dalton. Elastase is a simple enzyme containing no ions in the active center. Elastase consists of only one peptide chain and has a molecular weight<30,000 Dalton. Therefore, if both of these quite very different enzymes can be inhibited by acrylic acid polymers, such polymers are capable of acting systemically on very fundamental problems of skin disorder.

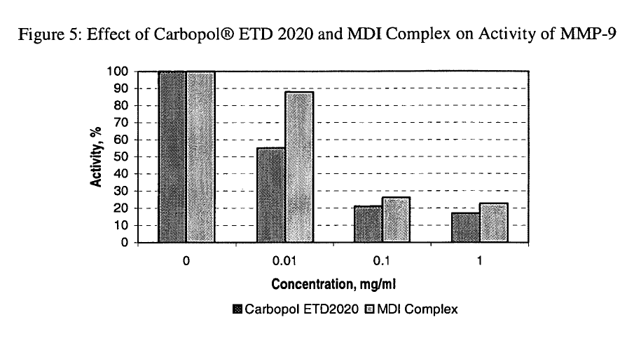

It was found that AAP products, such as carbomers, were able to demonstrate impressive MMP-9 inhibitory activity as shown in FIG. 5.

MMP-9 inhibition activity of AAPs was compared with the specific activity of matrix metalloproteinase enzyme inhibitors such as MDI Complex® (Atrium Biotechnologies, Inc., Canada), which is an acrylic acid polymer-free ingredient. Thus control experiments showed that Carbopol® ETD 2020 has IC₅₀=0.19 μg dry matter/ml while MDI Complex® demonstrates IC₅₀=4.2 μg dry matter/ml. Carbomers showed almost 20 times greater enzyme inhibition than MDI Complex.

The comparison of inhibitory activities demonstrated by carbomer and specific inhibitors is presented in Table 6.

TABLE 6
IC₅₀ Values of Carbomer and Enzyme Inhibitors.

| | Elastase | MMP-9 |
|---|---|---|
| Inhibitor | Inhibition* | Inhibition* |
| Carbopol ® ETD | 1.0 | 0.19 |
| 2020 | | |
| Elhibin ® | 3.5 | 41.0 |
| MDI Complex ® | 42.0 | 4.20 |

*IC₅₀ μg/ml

It was found that MMP-9 inhibition properties of acrylic acid polymers may depend on concentration of electrolyte. Thus at high concentration (1.0 M NaCl) inhibitory effect of AAPs is completely eliminated. Though not wishing to be bound by any particular theory, it is thought that electrostatic interaction between enzyme and polar groups of AAPs may be responsible for the inhibition of tested polymers. It should be noted, that effects of 1.0 M concentration of electrolyte is significant only for demonstrating the nature of inhibitory mechanism, since they involve the usage of nonphysiological conditions. The physiological concentration is 0.15 M is much lower than 1.0 M concentration of electrolyte required to eliminate the inhibitory effect of AAPs. Thus at physiological conditions acrylic acid polymers can effectively inhibit MMP-9.

The MMP-9 inhibition activity of AAPs could be compared with specific activity of MMP-9 inhibitors such as MDI Complex® (Atrium Biotechnologies, Inc., Canada). It was found that inhibitory effect of MDI Complex® was completely eliminated at 1.0 M concentration of electrolyte. It appeared that the inhibitory effects of both AAPs and MDI Complex® on MMP-9 are reversible.

### Example 4

The following example illustrates the use of AAP in emulsion representing sensitive skin facial moisturizer. It is recommended to use after sun exposure and for Rosacea conditions.

The emulsion consisting of:

| | % wt. |
|---|---|
| Water Phase | |
| Purified Water (q.s. to 100%) | 70.54 |
| Acrylates/C10-30 Alkyl Acrylate Crosspolymer | 0.01 |
| Glycerin | 7.50 |
| Phenonip | 0.20 |
| Oil Phase | |
| Isopropyl Myristate | 18.50 |
| Polysorbate 80 | 1.50 |
| Span 80 | 0.50 |
| Cetyl Alcohol | 3.00 |
| Stearyl Alcohol | 3.50 |
| Arlacel 165 (Glyceryl Stearate and PEG100 Stearate) | 4.50 |
| Dimethicone | 0.25 |
| | 100.00 |

Preparation procedure includes the heating of both phases to 80° C. and emulsification oil into water with high sheer mixing. The mix should be cooled slowly to 25° C. with continued mixing. The emulsion must be shaken well before use.

### Example 5

The following example illustrates the use of AAP in protectant gel. It is recommended to use to protect skin against insect bites. The gel consisting of:

| | % wt. |
|---|---|
| Phase A | |
| Purified Water (q.s. to 100%) | 73.05 |
| Pentylene Glycol | 10.00 |
| Ethoxydigidroglycol | 5.00 |
| Allantoin | 0.50 |
| Aloe Vera Extract | 0.25 |
| Phenonip | 0.20 |
| Phase B | |
| Carbomer | 0.01 |
| Phase C | |
| Hydroxypropylcellulose | 1.00 |
| Phase D | |
| SDA Alcohol 3A | 10.00 |
| | 100.00 |

Preparation procedure includes sprinkle Phase B to Phase A with high speed mixing. Heat to 65° C. with continued high speed mixing, and add Phase C. Mix for 30 minutes and cool to 30° C. Add Phase D and cool to room temperature.

### Example 6

The following example illustrates the use of AAP in spray. It is recommended to use as scalp anti-itch spray.

The gel consisting of:

| | % wt. |
|---|---|
| Phase A | |
| Purified Water (q.s. to 100%) | 54.94 |
| 1-3 Butylene Glycol | 4.00 |
| Sodium Polyacrylate | 0.01 |
| Phase B | |
| SDA Alcohol 3A | 40.00 |
| Hydrocortisone | 1.00 |
| Fragrance | 0.05 |
| | 100.00 |

Preparation procedure includes mixing of Phase A ingredients and parallel mixing Phase B ingredients. Then Phase A and Phase B are mixed until uniform.

### LIST OF REFERENCES

1. “Molecular weight of Carbopol® and Pemulen® polymers”, Noveon, Inc., 2001, TDS 222.
2. “Toxicity of the Carbopol® resins as a class”, Noveon, Inc., 2001, TDS 93.
3. “Application technology for Carbopol® resins and cosmetic formulations”, Noveon, Inc., 2001, TDS 60.
4. Lueβen H. L., Verhoef C. L., Borchard G. et al. Mucoadhesive polymers in peroral peptide drug delivery. II. Carbomer and polycarbophil are potent inhibitors of the intestinal proteolytic enzyme trypsin, Pharmaceutical Research, 12, pp. 1293-1298, 1995.
5. Lowe P. J., and Temple C. S., Calcitonin and insulin in isobutyl cyanoacrylate nanocapsules: protection against proteases and effect on intestinal absorption in rats, J Pharm Pharmacol 46 (1994) 547-552.
6. Bai J. P. F., Chang L. L., and Guo J. H. Effects of polyacrylic polymers on the lumenal proteolysis of peptide drugs in the colon. degradation of insulin and peptide drugs by chymotrypsin and trypsin. Journal of Pharmaceutical Sciences, 84, pp. 1291-1294, 1995.
7. Bai J. P. F., Chang L. L., and Guo J. H., Effects of poly(acrylic) polymers on the degradation of insulin and peptide drugs by chymotrypsin and trypsin, Journal of Pharmacy and Pharmacology, 48, pp. 17-21, 1996.
8. Ameye D., Voorspoels J., Foreman P., Tsai J., Richardson P., Geresh S. and Remon J. P. Trypsin inhibition, calcium and zinc ion binding of starch-g-poly(acrylic acid) copolymers and starch/poly(acrylic acid) mixtures for peroral peptide drug delivery. Journal of Controlled Release, 75 (3), pp. 357-364, 2001.
9. Valenta C., Marschutz M., Egyed C. and Bernkop-Schnurch A. Evaluation of the inhibition effect of thiolated poly(acrylates) on vaginal membrane bound aminopeptidase N and release of the model drug LH-RH. Journal of Pharmacy and Pharmacology, 54(5), pp. 603-610, 2002.
10. Strater N., Lipscomb W. N. Two-metal ion mechanism of bovine lens leucine aminopeptidase: active site solvent structure and binding mode of L-leucinal, a gem-diolate transition state analogue, by X-ray crystallography. Biochemistry, 34(45), pp. 12792-12800, 1995.
11. Madsen F. and Peppas N. A. Complexation graft copolymer networks: swelling properties, calcium binding and proteolytic enzyme inhibition. Biomaterials, 20(18), pp. 1701-1708, 1999.
12. Torres-Lugo M. and Peppas N. Transmucosal delivery systems for calcitonin: a review. Biomaterials, 21(12), pp.1191-1196, 2000.
13. Wiedow O., Wiese F., Streit V., Kalm C. and Cristophers E. Lesional elastase activity in psoriasis, contact dermatitis, and atopic dermatitis. Journal of Investigative Dermatology, 99, pp. 306-309, 1992.
14. Rogalski C., Meyer-Hoffert U., Proksch E., and Wiedow O. Human leukocyte elastase induces keratinocyte proliferation in vitro and in vivo. Journal of Investigative Dermatology., 118(1), pp. 49-54, 2002.
15. Skytt A., Stroemqvist M., and Egelrud T. Primary Substrate Specificity of Recombinant Human Stratum Corneum Chymotryptic Enzyme. Biochemical and Biophysical Research Communications, 211(2), p. 586, 1995.
16. Horikishi T., Igarashi S., Uchiwa H., Brysk H., and Brysk M. Role of endogenous cathepsin D-like and chymotrypsin-like proteolysis in human epidermal desquamation. British Journal of Dermatology, 141(3), pp. 453-459, 1999.
17. Olerud J. E., Usui M. L., Seckin D., Chiu D. S., Haycox C. L., Song I-S., Ansel J. C., Bunnett N. W. Neutral endopeptidase expression and distribution in human skin and wounds. Journal of Investigative Dermatology Symposium Proceedings, 112(6), pp. 873-881, 1999.
18. Ludolph-Hauser D., Schrubert C., and Wiedow O. Structural changes of human epidermis induced by human-derived proteases. Experimental Dermatology, 8(1), pp. 45-52, 1999.
19. Spenny M. L., Muangman P., Sullivan S. R., Bunnett N. W., Ansel J. C., Olerud J. E., and Gibran N. S. Neutral endopeptidase inhibition in diabetic wound repair. Wound Repair Regen, 2002, 10(5), pp. 295-301.
20. Pilcher B. K., Wang M., Qin X. J., Parks W. C., Senior R. W., and Welgus H. G. Role of matrix metalloproteinases and their inhibition in cutaneous wound healing and allergic contact hypersensitivity. Annals of the New York Academy of Sciences, 878, pp. 12-24, 1999.
21. Fräki J. Human skin proteases. Separation and characterization of two acid proteases resembling Cathepsin B1 and Cathepsin D and of an inhibitor of cathepsin B1. Archives of Dermatological Research, 225, pp. 317-330, 1976.
22. Lundstrom A. and Egelrud T. Stratum corneum chymotryptic enzyme: a proteinase which may be generally present in the Stratum corneum and with a possible involvement in desquamation. Acta. Derm. Venerol-Stockh. 71(6), pp. 471-474, 1991.
23. Watkinson A., Smith C. and Rawlings A. Identification and localization of tryptic and chymotryptic like enzymes in human Stratum corneum. Journal of Investigative Dermatology, 102(4), 637, 1994.
24. Marples R. Sex, constancy, and skin bacteria. Archives of Dermatological Research, 272, pp. 317-320, 1982.
25. Giogilli S. and Coll. Institute of Skin and Product Evaluation. 19^{th} National SICC congress in Italy, 1992.

1. A method for treating inflammatory conditions, comprising, administering a composition comprising at least one acrylic-acid based polymer in an amount that is effective in modulating the activity of at least one enzyme associated with inflammatory conditions.

2. The method of claim 1, wherein the at least one acrylic acid based polymer is a linear acrylic acid-based polymer, a cross-linked acrylic acid-based polymer, an high molecular weight cross-linked acrylic acid-based polymer, polymers of acrylic acid cross-linked with allyl sucrose, a polymer of acrylic acid cross-linked with allylpentaerythritol, a polymers of acrylic acid modified by long chain (C10-C30) acrylates, a polymers of acrylic acid modified by long chain (C10-C30) acrylates that are cross-linked with allylpentaerythritol, a copolymer of acrylic acid modified by long chain (C10-C30) alkyl acrylates, a copolymers of acrylic acid modified by long chain (C10-C30) alkyl acrylates cross-linked with allylpentaerythritol, a polymer of acrylic acid cross-linked with divinyl glycol, an homopolymer of acrylic acid cross-linked with an allyl ether of penaethritol, an allyl ether of sucrose or an allyl ether of propylene, a polyvinyl carboxy polymer, a carbomer, a copolymer of C-10 to C-30 alkyl acrylates and one or more monomers of acrylic acid, methacrylic acid or one of their simple esters cross-linked with an allyl ether of sucrose or an allyl ether of pentaerythritol, a graft copolymer with acrylic polymer backbone and dimethylpolysiloxane side chains, an hydrophilic/hydrophobic block copolymer such as an ammonium acylate or an acrylonitrogen copolymer, an acrylic and acrylonitrogen copolymer, an acrylic acid polyquaternium copolymer, a polyglycol, an hydrophobically modified ethylene oxide urethane, polymer or copolymer.

3. The method of claim 1, wherein in the compositions, the amount of the at least one acrylic-acid polymer is from about 0.001% wt to 95% wt.

4. The method of claim 1, wherein the at least one enzyme is peptide hydrolases, serine proteases, matrix metalloproteinases, collagenases, kinases, elastases or peroxydases.

5. The method of claim 1, wherein the inflammatory condition comprises skin reactions, allergic reactions, asthma, lung diseases or responses, kidney diseases, acute inflammatory diseases, vascular inflammatory disease, chronic inflammation, atherosclerosis, immune related diseases, angiopathy, myocarditis, nephritis, Crohn's disease, wound healing, arthritis, or type I or II diabetes and the associated vascular pathologies.

6. The method of claim 1, wherein the composition further comprises one or more formulation components including pharmaceutical excipient, preservative, emulsifier, emollient, rheology modifying agent, skin-feel additive, moisturizing agent, humectant, film former, pH adjuster/chelating agent, fragrance, effect pigment, color additive, water or combinations thereof.

7. The method of claim 1, wherein administering comprises applying the composition to the skin or other body surface.

8. The method of claim 1, wherein administering comprises applying the composition one or more time daily until the inflammatory condition subsides or ceases.

9. The method of claim 1, wherein the composition comprises an oil and water emulsion comprising 0.01% wt acrylic acid polymer, wherein the acrylic acid polymer is an acrylate/C10-30 alkyl acrylate crosspolymer.

10. A method of preventing inflammatory conditions, comprising, administering an amount of a one composition comprising at least one acrylic-acid based polymer that is effective in modulating the activity of at least one enzyme associated with inflammatory conditions.

11. The method of claim 10, wherein the at least one acrylic acid based polymer is a linear acrylic acid-based polymer, a cross-linked acrylic acid-based polymer, an high molecular weight cross-linked acrylic acid-based polymer, polymers of acrylic acid cross-linked with allyl sucrose, a polymer of acrylic acid cross-linked with allylpentaerythritol, a polymers of acrylic acid modified by long chain (C10-C30) acrylates, a polymers of acrylic acid modified by long chain (C10-C30) acrylates that are cross-linked with allylpentaerythritol, a copolymer of acrylic acid modified by long chain (C10-C30) alkyl acrylates, a copolymers of acrylic acid modified by long chain (C10-C30) alkyl acrylates cross-linked with allylpentaerythritol, a polymer of acrylic acid cross-linked with divinyl glycol, an homopolymer of acrylic acid cross-linked with an allyl ether of penaethritol, an allyl ether of sucrose or an allyl ether of propylene, a polyvinyl carboxy polymer, a carbomer, a copolymer of C-10 to C-30 alkyl acrylates and one or more monomers of acrylic acid, methacrylic acid or one of their simple esters cross-linked with an allyl ether of sucrose or an allyl ether of pentaerythritol, a graft copolymer with acrylic polymer backbone and dimethylpolysiloxane side chains, an hydrophilic/hydrophobic block copolymer such as an ammonium acylate or an acrylonitrogen copolymer, an acrylic and acrylonitrogen copolymer, an acrylic acid polyquaternium copolymer, a polyglycol, an hydrophobically modified ethylene oxide urethane, polymer or copolymer.

12. The method of claim 10, wherein in the compositions, the amount of the at least one acrylic-acid polymer is from about 0.001% wt to 95% wt.

13. The method of claim 10, wherein the at least one enzyme is peptide hydrolases, serine proteases, matrix metalloproteinases, collagenases, kinases, elastases or peroxydases.

14. The method of claim 10, wherein the inflammatory condition comprises skin reactions, allergic reactions, asthma, lung diseases or responses, kidney diseases, acute inflammatory diseases, vascular inflammatory disease, chronic inflammation, atherosclerosis, immune related diseases, angiopathy, myocarditis, nephritis, Crohn's disease, wound healing, arthritis, or type I or II diabetes and the associated vascular pathologies.

15. The method of claim 10, wherein the composition further comprises one or more formulation components including pharmaceutical excipient, preservative, emulsifier, emollient, rheology modifying agent, skin-feel additive, moisturizing agent, humectant, film former, pH adjuster/chelating agent, fragrance, effect pigment, color additive, water or combinations thereof.

16. The method of claim 10, wherein administering comprises applying the composition to the skin or other body surface.

17. The method of claim 10, wherein administering comprises applying the composition one or more time daily until the inflammatory condition subsides or ceases.

18. The method of claim 10, wherein the composition comprises an oil and water emulsion comprising 0.01% wt acrylic acid polymer, wherein the acrylic acid polymer is an acrylate/C10-30 alkyl acrylate crosspolymer.

19. A composition comprising, at least one acrylic acid based polymer, in an amount effective to modulate the activity of at least one enzyme associated with an inflammatory condition, wherein the amount of the acrylic acid based polymer is less than 95% wt of the composition.

20. The composition of claim 19, wherein the amount of the acrylic acid based polymer is less than 0.05% wt of the composition.
